(19) United States
(12) Patent Application Publication
(10) Pub. No.: US 2018/0105554 A1
(43) Pub. Date: Apr. 19, 2018

(54) USE OF DEXTRAN SULFATE TO ENHANCE PROTEIN A AFFINITY CHROMATOGRAPHY

(71) Applicant: BRISTOL-MYERS SQUIBB COMPANY, Princeton, NJ (US)

(72) Inventors: Mi JIN, West Chester, PA (US); Chao HUANG, Shrewsbury, MA (US); Zhengjian LI, Sudbury, MA (US); Zhijun TAN, Acton, MA (US)

(21) Appl. No.: 15/559,465

(22) PCT Filed: Mar. 18, 2016
(86) PCT No.: PCT/US2016/023073

§ 371 Date: Sep. 19, 2017

Related U.S. Application Data
(60) Provisional application No. 62/136,371, filed on Mar. 20, 2015.

(51) Int. Cl. C07K 1/22; C07K 16/06

(52) U.S. Cl. CPC C07K 1/22; C07K 16/065

(57) ABSTRACT

In certain embodiments, the invention provides a method of purifying a protein of interest from a mixture by using a dextran polymer.

### CROSS REFERENCE TO RELATED INVENTION

This application claims the benefit of U.S. provisional application Ser. No. 62/136,371 filed Mar. 20, 2015, hereby incorporated by reference in its entirety.

### BACKGROUND OF THE INVENTION

The large-scale, economic purification of proteins is an increasingly important problem for the biopharmaceutical industry. Therapeutic proteins are typically produced using prokaryotic or eukaryotic cell lines that are engineered to express the protein of interest from a recombinant plasmid containing the gene encoding the protein. Separation of the desired protein from the mixture of components fed to the cells and cellular by-products to an adequate purity, e.g., sufficient for use as a human therapeutic, poses a formidable challenge to biologics manufacturers.

Accordingly, there is a need in the art for alternative protein purification methods that can be used to expedite the large-scale processing of protein-based therapeutics, such as antibodies especially due to escalating high titers from cell culture.

### SUMMARY OF THE INVENTION

In certain embodiments, the present invention provides a method of purifying a protein of interest from a mixture which comprises the protein of interest and one or more contaminants, comprising: (a) adding a dextran polymer to the mixture under conditions suitable for the dextran polymer to bind to one or more contaminants, thereby to form a second mixture; (b) subjecting the second mixture to an affinity chromatography; (c) contacting the affinity chromatography with a wash solution; and (d) recovering the protein of interest in an elution solution, thereby purifying the protein of interest. Optionally, the second mixture does not have a significant precipitate. Optionally, the concentration of the dextran polymer is between about 0.01 and about 1 g/g protein in the mixture (e.g., between about 0.01 and about 0.5 g/g protein in the mixture). Optionally, the pH of the mixture is between about 6.5 and about 8.5 (e.g., between about 7.0 and about 8.0). Optionally, the temperature of the mixture is between about 15° C. and about 30° C. (e.g., between about 17° C. and about 27° C.). Optionally, the conductivity of the mixture is between about 13 mS/cm and about 22 mS/cm (e.g., between about 14.8 mS/cm and about 20.8 mS/cm).

In other embodiments, the present invention provides a method of purifying a protein of interest from a mixture which comprises the protein of interest and one or more contaminants, comprising: (a) subjecting the mixture to an affinity chromatography; (b) contacting the affinity chromatography with a wash solution which comprises a dextran polymer, under conditions suitable for the dextran polymer to bind to one or more contaminants; and (c) recovering the protein of interest in an elution solution, thereby purifying the protein of interest. Optionally, the concentration of the dextran polymer is between about 0.05 and about 2 g/L in the wash solution (e.g., between about 0.1 and about 1 g/L). Optionally, the pH of the wash solution is between about 5.0 and about 10.0 (e.g., between about 7.0 and about 8.0). Optionally, the wash solution comprises a salt, a detergent, and/or a chaotropic agent.

In certain specific embodiments, the contaminants are selected from host cell proteins, host cell metabolites, host cell constitutive proteins, nucleic acids, enzymes, endotoxins, viruses, product related contaminants, lipids, media additives and media derivatives, protein aggregates, chromatin, cell culture additives.

In certain specific embodiments, the dextran polymer is selected from dextran, dextran sulfate, dextran sulfate sodium salt, DEAE-dextran hydrochloride. For example, the molecular weight of dextran polymer ranges from 8 kDa to 500 kDa.

In certain specific embodiments, the mixture is selected from a cell culture, a harvested cell culture fluid, a cell culture supernatant, a conditioned cell culture supernatant, a cell lysate, and a clarified bulk. For example, the cell culture is a mammalian cell culture (e.g., a Chinese Hamster Ovary (CHO) cell culture) or a microbial cell culture. Optionally, the mixture comprises a feedstock. Optionally, the mixture comprises cell culture media into which the protein of interest is secreted. Optionally, the cell culture is in a bioreactor. Optionally, the protein of interest is substantially in the cell culture supernatant.

In certain specific embodiments, the affinity chromatography is a Protein A chromatography.

In certain specific embodiments, the methods further comprising subjecting the elution solution to a second chromatography (e.g., ion exchange, hydrophobic interaction, mimetic, and mixed mode).

In certain specific embodiments, the protein of interest is an antibody or an Fc fusion protein. For example, the antibody is a monoclonal antibody (e.g., a human, humanized and chimeric antibody).

In certain specific embodiments, the methods can be utilized to reduce the level of nucleic acids, host cell proteins, protein aggregates, and/or viruses in the elution solution.

### BRIEF DESCRIPTION OF THE DRAWING

FIG. 1 shows the impact of dextran sulfate treatment of clarified bulk (CB) on Protein A affinity chromatography performance, including HCP, DNA and step yield.

FIG. 2 shows the impact of dextran sulfate concentration in CB on Protein A affinity chromatography performance, including DNA, HCP and step yield.

FIG. 3 shows the impact of dextran sulfate wash buffer on Protein A affinity chromatography performance, including HCP, DNA and step yield.

FIG. 4 shows the effect of dextran sulfate in combination with salt, chaotropic agent, and detergent in wash buffer on Protein A affinity chromatography HCP, DNA and step yield.

FIG. 5 shows the impact of dextran sulfate in CB on PA step performance for mAb B.

FIG. 6 shows the impact of dextran sulfate in CB on PA step performance for mAb C.

FIG. 7 shows the impact of dextran sulfate in CB on PA step performance for mAb D.

FIG. 8 shows the impact of dextran sulfate in CB on PA step performance for Fc-B.

### DETAILED DESCRIPTION OF THE INVENTION

Affinity chromatography (e.g., Protein A affinity chromatography) is a standard platform for purifying monoclonal antibodies and Fc fusion proteins. In certain aspects, the present invention provides a method for enhancing protein purification by affinity chromatography through the addition of a dextran polymer (e.g., dextran sulfate) in the cell culture harvest (with cells), the clarified harvest (or clarified bulk), or the wash solution during affinity chromatography. Such methods have shown to effectively reduce one or more contaminants such as host cell proteins and/or DNAs and to enhance viral clearance. Such methods can be used as a robust downstream process for purifying proteins, such as monoclonal antibodies.

In certain embodiments, the present invention provides a method of purifying a protein of interest from a mixture which comprises the protein of interest and one or more contaminants, comprising: (a) adding a dextran polymer to the mixture under conditions suitable for the dextran polymer to bind to one or more contaminants, thereby to form a second mixture; (b) subjecting the second mixture to an affinity chromatography; (c) contacting the affinity chromatography with a wash solution; and (d) recovering the protein of interest in an elution solution, thereby purifying the protein of interest. Optionally, the second mixture does not have a significant precipitate. Optionally, the concentration of the dextran polymer is between about 0.01 and about 1 g/g protein in the mixture (e.g., between about 0.01 and about 0.5 g/g protein in the mixture). Optionally, the pH of the mixture is between about 6.5 and about 8.5 (e.g., between about 7.0 and about 8.0). Optionally, the temperature of the mixture is between about 15° C. and about 30° C. (e.g., between about 17° C. and about 27° C.). Optionally, the conductivity of the mixture is between about 13 mS/cm and about 22 mS/cm (e.g., between about 14.8 mS/cm and about 20.8 mS/cm).

In other embodiments, the present invention provides a method of purifying a protein of interest from a mixture which comprises the protein of interest and one or more contaminants, comprising: (a) subjecting the mixture to an affinity chromatography; (b) contacting the affinity chromatography with a wash solution which comprises a dextran polymer, under conditions suitable for the dextran polymer to bind to one or more contaminants; and (c) recovering the protein of interest in an elution solution, thereby purifying the protein of interest. Optionally, the concentration of the dextran polymer is between about 0.05 and about 2 g/L in the wash solution (e.g., between about 0.1 and about 1 g/L). Optionally, the pH of the wash solution is between about 5.0 and about 10.0 (e.g., between about 7.0 and about 8.0). Optionally, the wash solution comprises a salt, a detergent, and/or a chaotropic agent.

In certain specific embodiments, the contaminants are selected from host cell proteins, host cell metabolites, host cell constitutive proteins, nucleic acids, enzymes, endotoxins, viruses, product related contaminants, lipids, media additives and media derivatives, protein aggregates, chromatin, cell culture additives.

In certain specific embodiments, the dextran polymer is selected from dextran, dextran sulfate, dextran sulfate sodium salt, DEAE-dextran hydrochloride. For example, the molecular weight of dextran polymer ranges from 8 kDa to 500 kDa.

In certain specific embodiments, the mixture is selected from a cell culture, a harvested cell culture fluid, a cell culture supernatant, a conditioned cell culture supernatant, a cell lysate, and a clarified bulk. For example, the cell culture is a mammalian cell culture (e.g., a Chinese Hamster Ovary (CHO) cell culture) or a microbial cell culture. Optionally, the mixture comprises a feedstock. Optionally, the mixture comprises cell culture media into which the protein of interest is secreted. Optionally, the cell culture is in a bioreactor. Optionally, the protein of interest is substantially in the cell culture supernatant.

In certain specific embodiments, the affinity chromatography is a Protein A chromatography.

In certain specific embodiments, the methods further comprising subjecting the elution solution to a second chromatography (e.g., ion exchange, hydrophobic interaction, mimetic, and mixed mode).

In certain specific embodiments, the protein of interest is an antibody or an Fc fusion protein. For example, the antibody is a monoclonal antibody (e.g., a human, humanized and chimeric antibody).

In certain specific embodiments, the methods can be utilized to reduce the level of nucleic acids, host cell proteins, protein aggregates, and/or viruses in the elution solution.

### I. Definitions

In order that the present disclosure may be more readily understood, certain terms are first defined. As used in this application, except as otherwise expressly provided herein, each of the following terms shall have the meaning set forth below. Additional definitions are set forth throughout the application.

As used herein the term “dextran polymer” refers to dextran or any derivatives or its salt thereof, including, but not limited to, dextran, dextran sulfate, dextran sulfate sodium salt, and DEAE-dextran hydrochloride. For example, the molecular weight of dextran polymer may range from 8 kDa to 500 kDa.

As used herein, the term “protein of interest” is used in its broadest sense to include any protein (either natural or recombinant), present in a mixture, for which purification is desired. Such proteins of interest include, without limitation, hormones, growth factors, cyotokines, immunoglobulins (e.g., antibodies), and immunoglobulin-like domain-containing molecules (e.g., ankyrin or fibronectin domain-containing molecules).

As used herein, a “cell culture” refers to cells in a liquid medium. Optionally, the cell culture is contained in a bioreactor. The cells in a cell culture can be from any organism including, for example, bacteria, fungus, insects, mammals or plants. In a particular embodiment, the cells in a cell culture include cells transfected with an expression construct containing a nucleic acid that encodes a protein of interest (e.g., an antibody). Suitable liquid media include, for example, nutrient media and non-nutrient media. In a particular embodiment, the cell culture comprises a Chinese Hamster Ovary (CHO) cell line in nutrient media, not subject to purification by, for example, filtration or centrifugation.

As used herein, the term “clarified bulk” refers to a mixture from which particulate matter has been substantially removed. Clarified bulk includes cell culture, or cell lysate from which cells or cell debris has been substantially removed by, for example, filtration or centrifugation.

As used herein “bioreactor” takes its art recognized meaning and refers to a chamber designed for the controlled growth of a cell culture. The bioreactor can be of any size as long as it is useful for the culturing of cells, e.g., mammalian cells. Typically, the bioreactor will be at least 30 ml and may be at least 1, 10, 100, 250, 500, 1000, 2500, 5000, 8000, 10,000, 12,0000 liters or more, or any intermediate volume. The internal conditions of the bioreactor, including but not limited to pH and temperature, are typically controlled during the culturing period. A suitable bioreactor may be composed of (i.e., constructed of) any material that is suitable for holding cell cultures suspended in media under the culture conditions and is conductive to cell growth and viability, including glass, plastic or metal; the material(s) should not interfere with expression or stability of a protein of interest. One of ordinary skill in the art will be aware of, and will be able to choose, suitable bioreactors for use in practicing the present invention.

As used herein, a “mixture” comprises a protein of interest (for which purification is desired) and one or more contaminant, i.e., impurities. In one embodiment, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, the terms “separating” and “purifying” are used interchangeably, and refer to the selective removal of contaminants from a mixture containing a protein of interest (e.g., an antibody), for example using common industrial methods such as centrifugation or filtration. This separation results in the recovery of a mixture with a substantially reduced level of contaminants, and thereby serves to increase the purity of the protein of interest (e.g., an antibody) in the mixture.

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA), host cell proteins, host cell metabolites, enzymes, endotoxins, viruses, product related contaminants, lipids, media additives and media derivatives, protein aggregates, chromatin, or cell culture additives. Host cell contaminant proteins include, without limitation, those naturally or recombinantly produced by the host cell, as well as proteins related to or derived from the protein of interest (e.g., proteolytic fragments) and other process related contaminants.

As used herein “centrifugation” is a process that involves the use of the centrifugal force for the sedimentation of heterogeneous mixtures with a centrifuge, used in industry and in laboratory settings. This process is used to separate two immiscible liquids. For example, centrifugation can be used to remove certain contaminants from a mixture, including without limitation, a cell culture or clarified cell culture supernatant or capture-column captured elution pool.

As used herein “sterile filtration” is a filtration method that uses membrane filters, which are typically a filter with pore size 0.2 μm to effectively remove microorganisms or small particles. For example, sterile filtration can be used to remove certain contaminants from a mixture, including without limitation, a cell culture or clarified cell culture supernatant or capture-column captured elution pool.

As used herein “depth filtration” is a filtration method that uses depth filters, which are typically characterized by their design to retain particles due to a range of pore sizes within a filter matrix. The depth filter's capacity is typically defined by the depth, e.g., 10 inch or 20 inch of the matrix and thus the holding capacity for solids. For example, depth filtration can be used to remove certain contaminants from a mixture, including without limitation, a cell culture or clarified cell culture supernatant or capture-column captured elution pool.

As used herein, the term “tangential flow filtration” refers to a filtration process in which the sample mixture circulates across the top of a membrane, while applied pressure causes certain solutes and small molecules to pass through the membrane. For example, tangential flow filtration can be used to remove certain contaminants from a mixture, including without limitation, a cell culture or clarified cell culture supernatant or capture-column captured elution pool.

As used herein the term “chromatography” refers to the process by which a solute of interest, e.g., a protein of interest, in a mixture is separated from other solutes in the mixture by percolation of the mixture through an adsorbent, which adsorbs or retains a solute more or less strongly due to properties of the solute, such as pI, hydrophobicity, size and structure, under particular buffering conditions of the process. In a method of the present invention, chromatography can be used to remove contaminants from a mixture, including without limitation, a cell culture or clarified cell culture supernatant or capture-column captured elution pool.

The term “affinity chromatography” refers to a chromatographic method in which a biomolecule such as a polypeptide is separated based on a specific reversible interaction between the polypeptide and a binding partner covalently coupled to the solid phase. Examples of affinity interactions include, but are not limited to, the reversible interaction between an antigen and antibody, enzyme and substrate, or receptor and ligand. In certain specific embodiments, affinity chromatography involves the use of microbial proteins, such as Protein A or Protein G. Protein A is a bacterial cell wall protein that binds to mammalian IgGs primarily through their Fc regions. Protein A resin is useful for affinity purification and isolation of a variety antibody isotypes, particularly IgG1, IgG2, and IgG4. There are many Protein A resins available that are suitable for use in the purification process described herein. The resins are generally classified based on their backbone composition and include, for example, glass or silica-based resins; agarose-based resins; and organic polymer based resins.

The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange, anion exchange, and mixed mode chromatographies.

The phrase “ion exchange material” refers to a solid phase that is negatively charged (i.e., a cation exchange resin or membrane) or positively charged (i.e., an anion exchange resin or membrane). In one embodiment, the charge can be provided by attaching one or more charged ligands (or adsorbents) to the solid phase, e.g., by covalent linking. Alternatively, or in addition, the charge can be an inherent property of the solid phase (e.g., as is the case for silica, which has an overall negative charge).

A “cation exchange resin” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP Sepharose Fast Flow™, SP Sepharose High Performance from GE Healthcare, Toyopearl SP-650S and SP-650M from Tosoh, Macro-Prep High S from BioRad, Ceramic HyperD S, Trisacryl M and LS SP and Spherodex LS SP from Pall Technologies); a sulfoethyl based group (e.g., Fractogel SE, from EMD, Poros S-10 and S-20 from Applied Biosystems); a sulphopropyl based group (e.g., TSK Gel SP 5PW and SP-5PW-HR from Tosoh, Poros HS-20 and HS 50 from Applied Biosystems); a sulfoisobutyl based group (e.g., Fractogel EMD SO3⁻ from EMD); a sulfoxyethyl based group (e.g., SE52, SE53 and Express-Ion S from Whatman), a carboxymethyl based group (e.g., CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrx Cellufine C500 and C200 from Millipore, CM52, CM32, CM23 and Express-Ion C from Whatman, Toyopearl CM-650S, CM-650M and CM-650C from Tosoh); sulfonic and carboxylic acid based groups (e.g., BAKERBOND Carboxy-Sulfon from J.T. Baker); a carboxylic acid based group (e.g., WP CBX from J.T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO—from EMD); a sulfonic acid based group (e.g., Hydrocell SP from Biochrom Labs Inc., DOWEX Fine Mesh Strong Acid Cation Resin from Dow Liquid Separations, UNOsphere S, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (e.g., P11 from Whatman).

An “anion exchange resin” refers to a solid phase which is positively charged, thus having one or more positively charged ligands attached thereto. Any positively charged ligand attached to the solid phase suitable to form the anionic exchange resin can be used, such as quaternary amino groups Commercially available anion exchange resins include DEAE cellulose, Poros PI 20, PI 50, HQ 10, HQ 20, HQ 50, D 50 from Applied Biosystems, Sartobind Q from Sartorius, MonoQ, MiniQ, Source 15Q and 30Q, Q, DEAE and ANX Sepharose Fast Flow, Q Sepharose high Performance, QAE SEPHADEX™ and FAST Q SEPHAROSE™ (GE Healthcare), WP PEI, WP DEAM, WP QUAT from J.T. Baker, Hydrocell DEAE and Hydrocell QA from Biochrom Labs Inc., UNOsphere Q, Macro-Prep DEAE and Macro-Prep High Q from Biorad, Ceramic HyperD Q, ceramic HyperD DEAE, Trisacryl M and LS DEAE, Spherodex LS DEAE, QMA Spherosil LS, QMA Spherosil M and Mustang Q from Pall Technologies, DOWEX Fine Mesh Strong Base Type I and Type II Anion Resins and DOWEX MONOSPHER E 77, weak base anion from Dow Liquid Separations, Intercept Q membrane, Matrex Cellufine A200, A500, Q500, and Q800, from Millipore, Fractogel EMD TMAE, Fractogel EMD DEAE and Fractogel EMD DMAE from EMD, Amberlite weak strong anion exchangers type I and II, DOWEX weak and strong anion exchangers type I and II, Diaion weak and strong anion exchangers type I and II, Duolite from Sigma-Aldrich, TSK gel Q and DEAE 5PW and 5PW-HR, Toyopearl SuperQ-650S, 650M and 650C, QAE-550C and 650S, DEAE-650M and 650C from Tosoh, QA52, DE23, DE32, DE51, DE52, DE53, Express-Ion D and Express-Ion Q from Whatman, and Sartobind Q (Sartorius corporation, New York, USA).

A “mixed mode ion exchange resin” or “mixed mode” refers to a solid phase which is covalently modified with cationic, anionic, and/or hydrophobic moieties. Examples of mixed mode ion exchange resins include Capto MMC and Capto adhere (GE Healthcare, Uppsala, Sweden), BAKERBOND ABX™ (J. T. Baker; Phillipsburg, N.J.), ceramic hydroxyapatite type I and II and fluoride hydroxyapatite (BioRad; Hercules, Calif.) and MEP and MBI HyperCel (Pall Corporation; East Hills, N.Y.).

A “hydrophobic interaction chromatography resin” refers to a solid phase which is covalently modified with phenyl, octyl, or butyl chemicals. Hydrophobic interaction chromatography is a separation technique that uses the properties of hydrophobicity to separate proteins from one another. In this type of chromatography, hydrophobic groups such as, phenyl, octyl, hexyl or butyl are attached to the stationary column. Proteins that pass through the column that have hydrophobic amino acid side chains on their surfaces are able to interact with and bind to the hydrophobic groups on the column. Examples of hydrophobic interaction chromatography resins include Phenyl sepharose FF, Capto Phenyl (GE Healthcare, Uppsala, Sweden), Phenyl 650-M (Tosoh Bioscience, Tokyo, Japan) and Sartobind Phenyl (Sartorius corporation, New York, USA).

### II. Proteins of Interest

In certain aspects, methods of the present invention may be used to purify any protein of interest including, but not limited to, proteins having pharmaceutical, diagnostic, agricultural, and/or any of a variety of other properties that are useful in commercial, experimental or other applications. In addition, a protein of interest can be a protein therapeutic. In certain embodiments, proteins purified using methods of the present invention may be processed or modified. For example, a protein of interest in accordance with the present invention may be glycosylated.

Thus, the present invention may be used to culture cells for production of any therapeutic protein, such as pharmaceutically or commercially relevant enzymes, receptors, receptor fusion proteins, antibodies (e.g., monoclonal or polyclonal antibodies), antigen-binding fragments of an antibody, Fc fusion proteins, cytokines, hormones, regulatory factors, growth factors, coagulation/clotting factors, or antigen-binding agents. The above list of proteins is merely exemplary in nature, and is not intended to be a limiting recitation. One of ordinary skill in the art will know that other proteins can be produced in accordance with the present invention, and will be able to use methods disclosed herein to produce such proteins.

In one particular embodiment of the invention, the protein purified using the method of the invention is an antibody. The term “antibody” is used in the broadest sense to cover monoclonal antibodies (including full length monoclonal antibodies), polyclonal antibodies, multispecific antibodies (e.g., bispecific antibodies), antibody fragments, immunoadhesins and antibody-immunoadhesin chimerias.

An “antibody fragment” includes at least a portion of a full length antibody and typically an antigen binding or variable region thereof. Examples of antibody fragments include Fab, Fab′, F(ab′)₂, and Fv fragments; single-chain antibody molecules; diabodies; linear antibodies; and multispecific antibodies formed from engineered antibody fragments.

The term “monoclonal antibody” is used in the conventional sense to refer to an antibody obtained from a population of substantially homogeneous antibodies such that the individual antibodies comprising the population are identical except for possible naturally occurring mutations that may be present in minor amounts. Monoclonal antibodies are highly specific, being directed against a single antigenic site. This is in contrast with polyclonal antibody preparations which typically include varied antibodies directed against different determinants (epitopes) of an antigen, whereas monoclonal antibodies are directed against a single determinant on the antigen. The term “monoclonal”, in describing antibodies, indicates the character of the antibody as being obtained from a substantially homogeneous population of antibodies, and is not to be construed as requiring production of the antibody by any particular method. For example, monoclonal antibodies used in the present invention can be produced using conventional hybridoma technology first described by Kohler et al., Nature 256:495 (1975), or they can be made using recombinant DNA methods (see, e.g., U.S. Pat. No. 4,816,567). Monoclonal antibodies can also be isolated from phage antibody libraries, e.g., using the techniques described in Clackson et al., Nature 352:624-628 (1991); Marks et al., J. Mol. Biol. 222:581-597 (1991); and U.S. Pat. Nos. 5,223,409; 5,403,484; 5,571,698; 5,427,908 5,580,717; 5,969,108; 6,172,197; 5,885,793; 6,521,404; 6,544,731; 6,555,313; 6,582,915; and 6,593,081).

The monoclonal antibodies described herein include “chimeric” and “humanized” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). “Humanized” forms of non-human (e.g., murine) antibodies are chimeric antibodies which contain minimal sequence derived from non-human immunoglobulin. For the most part, humanized antibodies are human immunoglobulins (recipient antibody) in which the hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

The monoclonal antibodies described herein also include “human” antibodies, which can be isolated from various sources, including, e.g., from the blood of a human patient or recombinantly prepared using transgenic animals. Examples of such transgenic animals include KM-Mouse® (Medarex, Inc., Princeton, N.J.) which has a human heavy chain transgene and a human light chain transchromosome (see WO 02/43478), Xenomouse® (Abgenix, Inc., Fremont Calif.; described in, e.g., U.S. Pat. Nos. 5,939,598; 6,075,181; 6,114,598; 6, 150,584 and 6,162,963 to Kucherlapati et al.), and HuMAb-Mouse® (Medarex, Inc.; described in, e.g., Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, U.S. Pat. Nos. 5,545,806; 5,569,825; 5,625,126; 5,633,425; 5,789,650; 5,877,397; 5,661,016; 5,814,318; 5,874,299; and U.S. Pat. Nos. 5,770,429; 5,545,807; and PCT Publication Nos. WO 92/03918, WO 93/12227, WO 94/25585, WO 97/13852, WO 98/24884 and WO 99/45962, WO 01/14424 to Korman et al.). Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### III. Mixtures Containing a Protein of Interest

The methods of the invention can be applied to any mixture containing a protein of interest. In one embodiment, the mixture is obtained from or produced by living cells that express the protein to be purified (e.g., naturally or by genetic engineering). Optionally, the cells in a cell culture include cells transfected with an expression construct containing a nucleic acid that encodes a protein of interest. Methods of genetically engineering cells to produce proteins are well known in the art. See e.g., Ausabel et al., eds. (1990), Current Protocols in Molecular Biology (Wiley, New York) and U.S. Pat. Nos. 5,534,615 and 4,816,567, each of which are specifically incorporated herein by reference. Such methods include introducing nucleic acids that encode and allow expression of the protein into living host cells. These host cells can be bacterial cells, fungal cells, insect cells or, preferably, animal cells grown in culture. Bacterial host cells include, but are not limited to E. coli cells. Examples of suitable E. coli strains include: HB101, DH5α, GM2929, JM109, KW251, NM538, NM539, and any E. coli strain that fails to cleave foreign DNA. Fungal host cells that can be used include, but are not limited to, Saccharomyces cerevisiae, Pichia pastoris and Aspergillus cells. Insect cells that can be used include, but are not limited to, Bombyx mori, Mamestra drassicae, Spodoptera frugiperda, Trichoplusia ni, Drosophilia melanogaster.

A number of mammalian cell lines are suitable host cells for expression of proteins of interest. Mammalian host cell lines include, for example, COS, PER.C6, TM4, VERO076, DXB11, MDCK, BRL-3A, W138, Hep G2, MMT, MRC 5, FS4, CHO, 293T, A431, 3T3, CV-1, C3H10T½, Colo205, 293, HeLa, L cells, BHK, HL-60, FRhL-2, U937, HaK, Jurkat cells, Rat2, BaF3, 32D, FDCP-1, PC12, M1x, murine myelomas (e.g., SP2/0 and NS0) and C2C12 cells, as well as transformed primate cell lines, hybridomas, normal diploid cells, and cell strains derived from in vitro culture of primary tissue and primary explants. New animal cell lines can be established using methods well known by those skilled in the art (e.g., by transformation, viral infection, and/or selection). Any eukaryotic cell that is capable of expressing the protein of interest may be used in the disclosed cell culture methods. Numerous cell lines are available from commercial sources such as the American Type Culture Collection (ATCC). In one embodiment of the invention, the cell culture, e.g., the large-scale cell culture, employs hybridoma cells. The construction of antibody-producing hybridoma cells is well known in the art. In one embodiment of the invention, the cell culture, e.g., the large-scale cell culture, employs CHO cells to produce the protein of interest such as an antibody (see, e.g., WO 94/11026). Various types of CHO cells are known in the art, e.g., CHO-K1, CHO-DG44, CHO-DXB11, CHO/dhfr⁻ and CHO-S.

In a specific embodiment, methods of the present invention comprise effectively removing contaminants from a mixture (e.g., a cell culture, cell lysate or clarified bulk) which contains a high concentration of a protein of interest (e.g., an antibody). For example, the concentration of a protein of interest may range from about 0.5 to about 50 mg/ml (e.g., 0.5, 1, 5, 10, 15, 20, 25, 30, 35, 40, 45 or 50 mg/ml).

Preparation of mixtures initially depends on the manner of expression of the protein. Some cell systems directly secrete the protein (e.g., an antibody) from the cell into the surrounding growth media, while other systems retain the antibody intracellularly. For proteins produced intracellularly, the cell can be disrupted using any of a variety of methods, such as mechanical shear, osmotic shock, and enzymatic treatment. The disruption releases the entire contents of the cell into the homogenate, and in addition produces subcellular fragments which can be removed by centrifugation or by filtration. A similar problem arises, although to a lesser extent, with directly secreted proteins due to the natural death of cells and release of intracellular host cell proteins during the course of the protein production run.

In one embodiment, cells or cellular debris are removed from the mixture, for example, to prepare clarified bulk. The methods of the invention can employ any suitable methodology to remove cells or cellular debris. If the protein is produced intracellularly, as a first step, the particulate debris, either host cells or lysed fragments, can be removed, for example, by a centrifugation or filtration step in order to prepare a mixture which is then subjected to purification according the methods described herein (i.e., from which a protein of interest is purified). If the protein is secreted into the medium, the recombinant host cells may be separated from the cell culture medium by, e.g., centrifugation, tangential flow filtration or depth filtration, in order to prepare a mixture from which a protein of interest is purified.

In another embodiment, cell culture or cell lysate is used directly without first removing the host cells. The methods of the invention may be suited to using mixtures comprising a secreted protein and a suspension of host cells.

### IV. Addition of Dextran Polymer to a Mixture or a Wash Solution

In certain aspects, methods of the present invention involve adding a dextran polymer (e.g., dextran sulfate) in the cell culture harvest (with cells), the clarified harvest (or clarified bulk), or the wash solution during affinity chromatography. The dextran polymer may be selected from dextran, dextran sulfate, dextran sulfate sodium salt, DEAE-dextran hydrochloride. For example, the molecular weight of dextran polymer ranges from 8 kDa to 500 kDa.

In certain embodiments, the method of the present invention comprises: (a) adding a dextran polymer to the mixture under conditions suitable for the dextran polymer to bind to one or more contaminants, thereby to form a second mixture; (b) subjecting the second mixture to an affinity chromatography; (c) contacting the affinity chromatography with a wash solution; and (d) recovering the protein of interest in an elution solution, thereby purifying the protein of interest.

The concentration of the dextran polymer in the mixture can be determined empirically for each protein mixture using methods described herein. For example, the concentration of the dextran polymer is between about 0.01 and about 1 g/g protein in the mixture (e.g., between about 0.01 and about 0.5 g/g protein in the mixture). The pH of the mixture can be determined empirically for each protein mixture using methods described herein. For example, the pH of the mixture is between about 6.5 and about 8.5 (e.g., between about 7.0 and about 8.0). The temperature of the mixture can be determined empirically for each protein mixture using methods described herein. For example, the temperature of the mixture is between about 15° C. and about 30° C. (e.g., between about 17° C. and about 27° C.). The conductivity of the mixture can be determined empirically for each protein mixture using methods described herein. For example, the conductivity of the mixture is between about 13 mS/cm and about 22 mS/cm (e.g., between about 14.8 mS/cm and about 20.8 mS/cm).

Optionally, the dextran polymer is added to the mixture and mixed for a particular length of time. The optimum length of mixing required to facilitate binding of the dextran polymer to one or more contaminants can be determined empirically for each protein mixture using methods described herein. Preferably the mixing time is greater than about 5 minutes (e.g., about 5, 10, 15, 20, 30, 60, 90, 120, 240, or 480 minutes).

In other embodiments, the method of the present invention comprises: (a) subjecting the mixture to an affinity chromatography; (b) contacting the affinity chromatography with a wash solution which comprises a dextran polymer, under conditions suitable for the dextran polymer to bind to one or more contaminants; and (c) recovering the protein of interest in an elution solution, thereby purifying the protein of interest.

The concentration of the dextran polymer in the wash solution can be determined empirically for each protein of interest and/or each wash solution using methods described herein. For example, the concentration of the dextran polymer is between about 0.05 and about 2 g/L in the wash solution (e.g., between about 0.1 and about 1 g/L). The pH of the wash solution can be determined empirically for each protein of interest and/or each wash solution using methods described herein. For example, the pH of the wash solution is between about 5.0 and about 10.0 (e.g., between about 7.0 and about 8.0). Optionally, the wash solution comprises a salt, a detergent, and/or a chaotropic agent.

The present disclosure is further illustrated by the following examples, which should not be construed as further limiting. The contents of all figures and all references, patents and published patent applications cited throughout this application are expressly incorporated herein by reference in their entireties.

### Example 1

### Effect of Dextran Sulfate Treatment of CB on Fc Fusion Protein A Purification

1a. Dextran Sulfate Addition in Clarified Bulk Reduces ProA Pool HCP and DNA

This experiment evaluated the impact of dextran sulfate (DS) treatment of clarified bulk (CB) on DNA and HCP reduction over the Protein A affinity chromatography step for an Fc fusion protein (Fc-A).

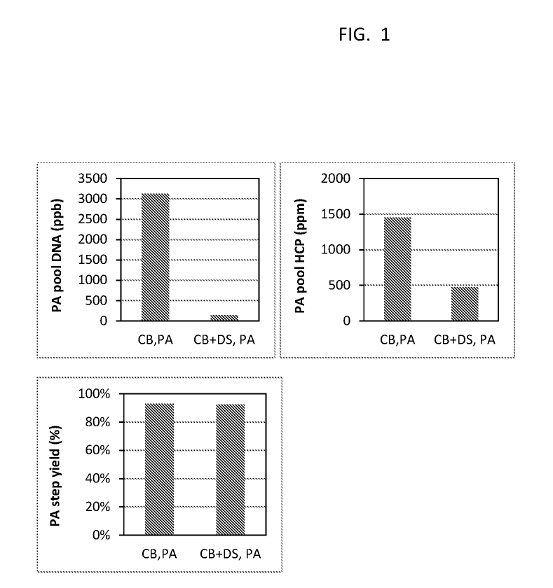

The dextran sulfate was added to Fc-A CB to a final concentration of 0.1 g/g _{Protein} (g/g ₚᵣₒₜₑᵢₙ: gram dextran sulfate per gram of protein product in the CB). The CB with dextran sulfate addition (CB+DS) was then stirred for >15 minutes at room temperature. No visible precipitation was observed upon dextran sulfate addition or subsequent agitation. The CB+DS was then used as the load material for a subsequent Protein A affinity chromatography (PA) step. The PA column operating conditions were previously optimized for impurity reduction. Untreated CB was used as the load material for a control PA run. The PA elution pool impurity levels and yield are shown in FIG. 1. CB treatment with Dextran sulfate at 0.1 g/g _{Protein} concentration significantly reduced DNA, HCP in the PA elution pool compared with CB without treatment. Similar step yield was achieved for CB treated and untreated runs.

1b. Dextran Sulfate Concentration Effect on ProA Pool DNA Reduction

This experiment evaluated the impact of dextran sulfate concentration during CB treatment on DNA and HCP reduction over the PA step for Fc-A.

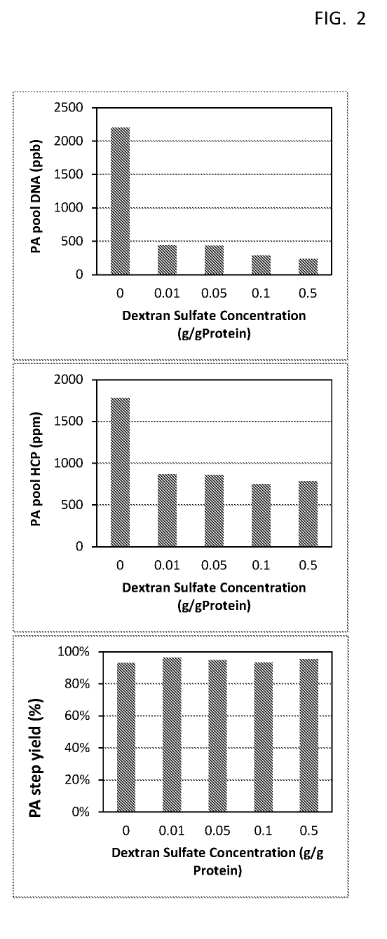

Dextran sulfate final concentration was varied in the range from 0.01 g/g ₚᵣₒₜₑᵢₙ to 0.5 g/g ₚᵣₒₜₑᵢₙ in the CB. CB without dextran sulfate treatment was included in the experiment as a control. All procedures and operating conditions were the same as described in example 1a. The PA elution pool impurity levels and yield are shown in FIG. 2. CB treatment with Dextran sulfate in the concentration range from 0.01 g/g _{Protein} to 0.5 g/g _{Protein} significantly reduced DNA and HCP in the PA elution pool compared to CB without treatment. There is a concentration dependent effect for DNA reduction. Similar step yield was achieved for CB treated and untreated runs.

### Example 2

### Effect of Dextran Sulfate in PA Wash Buffer on Fc Fusion Protein A Purification

2a. Dextran Sulfate in PA Wash Buffer Reduced ProA Pool HCP and DNA

This experiment evaluated the impact of a dextran sulfate wash buffer on DNA and HCP reduction over the Protein A affinity chromatography step for an Fc fusion protein (Fc-A).

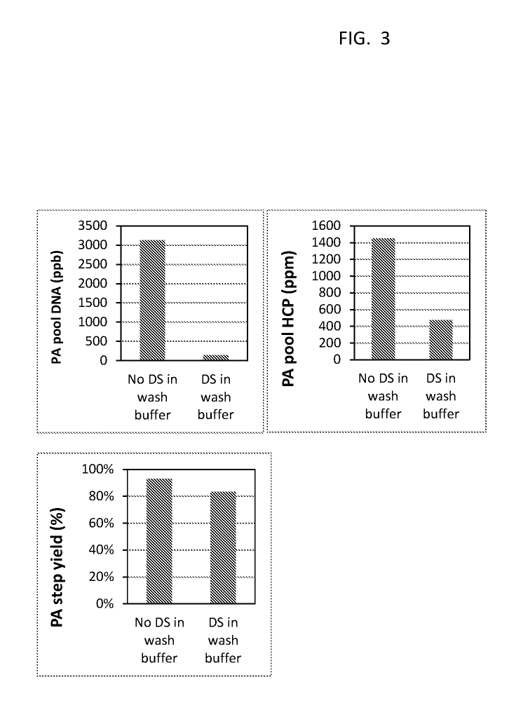

The Fc-A CB was purified using Protein A affinity chromatography. The protein A chromatography consists of the following major steps: equilibration, loading, multiple washes, elution, cleaning, re-equilibration, storage. In one experiment, the Protein A wash buffer for wash 3 step was 25 mM sodium phosphate, 0.5 g/L dextran sulfate, pH7. In a control experiment, the Protein A wash buffer for wash 3 step was 25 mM sodium phosphate, pH7 without dextran sulfate. The PA elution pool impurity levels and yield are shown in FIG. 3. The experiment run with Dextran sulfate containing wash buffer showed reduced levels of DNA and HCP in the PA elution pool compared to the run using wash buffer without dextran sulfate. Slightly reduced step yield was observed for the run with dextran sulfate in the wash buffer.

2b. Combination of Salt, Detergent, Chaotropic Reagent with Dextran Sulfate in PA Wash Buffer

This experiment evaluated other wash buffer components, including salt (S), chaotropic agent (C) and detergent (D), in combination with dextran sulfate as PA wash buffers. The salt tested is sodium chloride, the chaotropic reagent tested is urea, the detergent tested is Triton X-100. The detailed wash buffer compositions tested were listed in Table 1. The run with salt, chaotropic agent, and detergent in wash buffer was used as control condition.

TABLE 1
Wash buffer composition to evaluate dextran sulfate effect
in combination with selected buffer components

| Run ID | Wash buffer composition |
|---|---|
| S + C + D | 25 mM sodium phosphate, 1M sodium chloride, |
| (control) | 2M Urea, 0.5% Triton X-100, pH 7 |
| S + D + DS | 25 mM sodium phosphate, 1M sodium chloride, |
| | 0.5% Triton X-100, 0.5 g/L dextran sulfate, pH 7 |
| S + C + | 25 mM sodium phosphate, 1M sodium chloride, |
| D + DS | 2M Urea, 0.5% Triton X-100, 0.5 g/L dextran sulfate, pH 7 |
| D + DS | 25 mM sodium phosphate, 0.5% Triton X-100, |
| | 0.5 g/L dextran sulfate, pH 7 |
| C + D + DS | 25 mM sodium phosphate, 2M Urea, 0.5% |
| | Triton X-100, 0.5 g/L dextran sulfate, pH 7 |

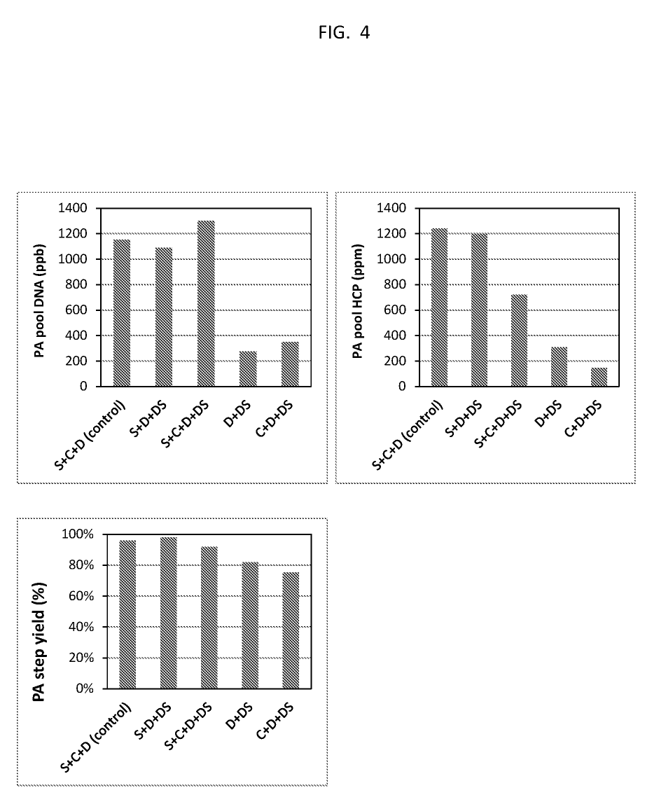

The PA elution pool impurity levels and yield are shown in FIG. 4. Experimental runs with wash buffers containing dextran sulfate but not high salt (D+DS, C+D+DS) resulted in significantly reduced DNA and HCP levels (>70%) in the PA elution pool compared to control condition (S+C+D). In comparison, experimental runs with wash buffers containing dextran sulfate in combination with high salt (S+D+DS, S+C+D+DS) similar levels of DNA compared to control condition. For HCP, S+C+D+DS showed 42% reduction in the PA elution pool compared to control condition (S+C+D), while S+D+DS showed similar level of HCP compared to the control. Improved impurity removal correlated with yield loss over the PA step. The data demonstrated dextran sulfate addition in PA wash buffer can improve PA impurity removal. High salt (e.g. 1M NaCl) in combination with dextran sulfate in wash buffer reduced dextran sulfate effectiveness.

### Example 3

### Effect of Dextran Sulfate Treatment of CB on the PA Purification of Multiple Monoclonal Antibodies and Fc Fusion Protein

The effect of dextran sulfate treatment of CB on the PA performance was further evaluated using multiple monoclonal antibodies (mAb) and another Fc fusion protein.

Dextran sulfate final concentration was varied in the range from 0.01 g/g ₚᵣₒₜₑᵢₙ to 1 g/g ₚᵣₒₜₑᵢₙ in the CB. CB without dextran sulfate treatment was included in the experiment as a control. The subsequent PA chromatography step used the platform operating condition with column loading optimized for each mAb or Fc-fusion protein. The PA elution pool impurity levels and yield were compared to the control experiment where no dextran sulfate was added to CB.

3a. Effect of Dextran Sulfate Treatment of CB on mAb B PA Performance

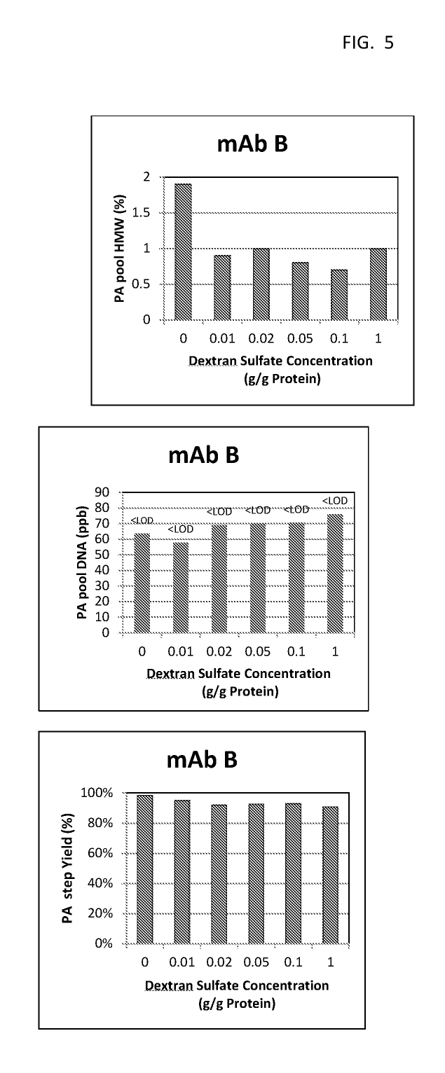

As shown in FIG. 5, for mAb B molecule, CB treatment with Dextran sulfate in the concentration range from 0.01 g/g _{Protein} to 1 g/g _{Protein} significantly reduced HMW levels in the PA elution pool compared to CB without dextran sulfate. PA pool DNA levels for all conditions were below assay limit of detection, therefore, they were not compared. PA step yields for all conditions were above 90%.

3b. Effect of Dextran Sulfate Treatment of CB on mAb C PA Performance

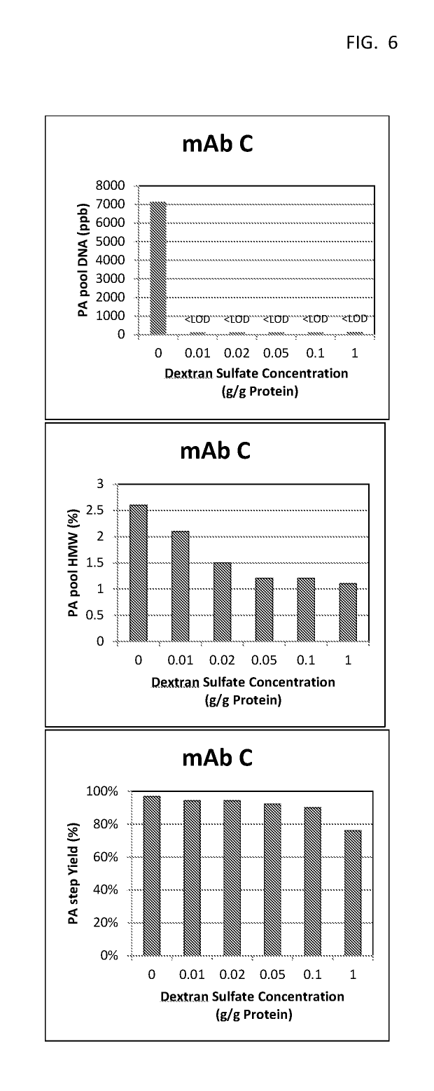

As shown in FIG. 6, for mAb C, CB treatment with dextran sulfate in the concentration range from 0.01 g/g _{Protein} to 1 g/g _{Protein} reduced HMW levels in the PA elution pool. In the concentration range between 0 to 0.05 g/g _{Protein}, the PA pool HMW reduced in a dextran sulfate concentration dependent manner. PA pool DNA levels for all dextran treated conditions were below assay limit of detection, significantly lower than that of no treated control condition. Dextran sulfate treatment showed minimal impact on PA step yield in the concentration range up to 0.1 g/g _{Protein}. At 1 g/g _{Protein}, PA step yield reduced from >90% to 76%.

3c. Effect of Dextran Sulfate Treatment of CB on mAb D PA Performance

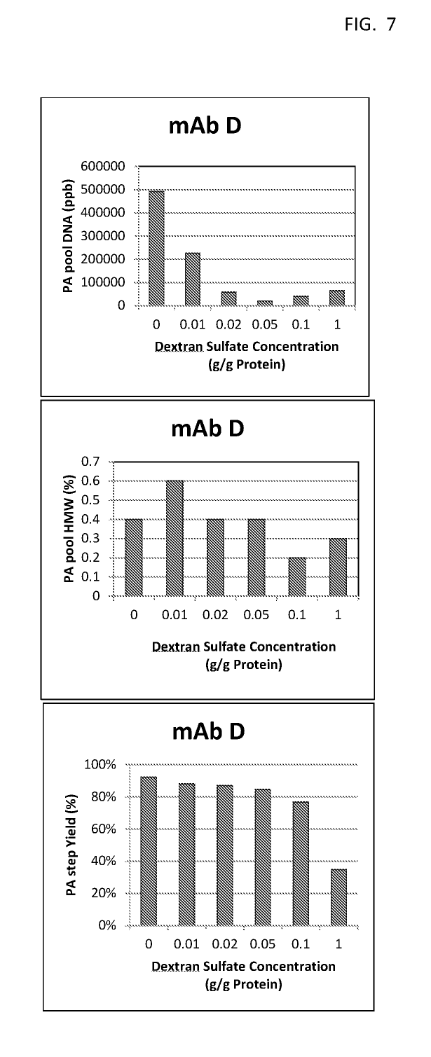

CB treatment with dextran sulfate was performed in the concentration range from 0.01 g/g _{Protein} to 1 g/g _{Protein}. Slight turbidity was observed after dextran sulfate addition, all material was filtered through 0.2 um filter prior to loading on to column. As shown in FIG. 7, for mAb D, CB treatment with Dextran sulfate in the concentration range from 0.01 g/g _{Protein} to 1 g/g _{Protein} reduced DNA levels in the PA elution pool. In the concentration range between 0 to 0.05 g/g _{Protein}, the PA pool DNA levels in PA pool reduced in a dextran sulfate concentration dependent manner, with lowest DNA level at DS concentration of 0.05 g/g _{Protein} to be 4% of that of the control condition. PA pool HMW levels for dextran treated conditions were not significantly different. Dextran sulfate treatment showed minimal impact on PA step yield in the concentration range up to 0.05 g/g _{Protein}. At 0.1 g/g _{Protein}, PA step yield reduced from 92% in control to 76%. At 1 g/g _{Protein}, PA step yield reduced to 34%.

3c. Effect of Dextran Sulfate Treatment of CB on Fc-Fusion Protein E PA Performance

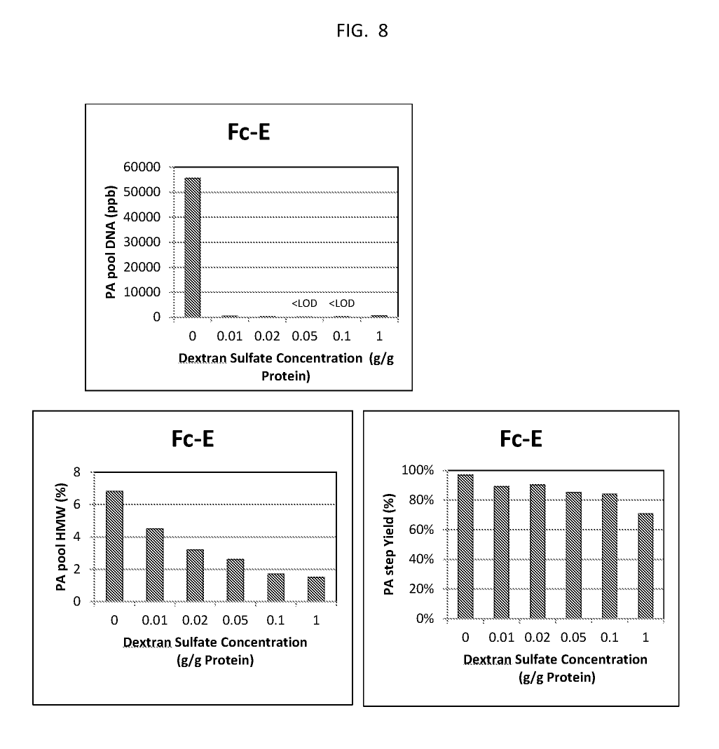

CB treatment with dextran sulfate was performed in the concentration range from 0.01 g/g _{Protein} to 1 g/g _{Protein}. Slight turbidity was observed after dextran sulfate addition, all material was filtered through 0.2 um filter prior to loading on to PA column. As shown in FIG. 8, for Fc-fusion protein E (Fc-E), CB treatment with Dextran sulfate in the concentration range from 0.01 g/g _{Protein} to 1 g/g _{Protein} significantly reduced DNA levels in the PA elution pool. HMW levels in the PA pool were also reduced in a concentration dependent in the dextran sulfate concentration range between 0 to 0.1 g/g _{Protein}. Dextran sulfate treatment showed minimal impact on PA step yield in the concentration range up to 0.1 g/g _{Protein}. At 1 g/g _{Protein}, PA step yield reduced from 97% in control to 71%.

### Example 4

### Effect of pH, Temperature and Conductivity on Dextran Sulfate Treatment of CB

The purpose of this set of experiments was to evaluate the process robustness for dextran sulfate treatment of CB. The effects of process parameters during dextran sulfate treatment of CB, including pH, temperature and solution conductivity, were evaluated in a full factorial design of experiment (DoE). The process parameter ranges were summarized in Table 2. The run condition matrix is shown in Table 3. Monoclonal antibody F (mAb F) was used in this set of experiments. The final dextran sulfate concentration in CB was 0.02 g/g _{Protein}. A control run without dextran sulfate addition in CB was also conducted. All PA runs used the same operating condition optimized for mAb F.

TABLE 2
Process parameter ranges evaluated in full factorial DoE of mAb F

| Process Parameters | | | |
|---|---|---|---|
| (Factors) | Low level (−1) | Center (0) | High level (+1) |
| CB Cond. (mS/cm) | 14.8 | 17.8 | 20.8 |
| CB pH | 7.3 | 7.8 | 8.3 |
| CB Temp (° C.) | 17 | 22 | 27 |

The data in Table 3 demonstrated that dextran sulfate treatment of CB consistently reduced the PA pool impurity levels. The average DNA level was 21±4 ppb for all 11 PA pools using dextran sulfated treated CB as PA load material, compared to 2349 ppb for untreated CB as PA load material. HCP, HMW and rPrA levels in PA pools were also lower for dextran sulfate treated runs than the untreated run. Analysis of DoE data also showed that temperature, pH and solution conductivity in the tested range didn't significantly impact the dextran sulfate treatment of CB and the subsequent PA performance, demonstrating a robust operating range for dextran sulfate treatment of CB.

### Example 5

### Effect of Dextran Sulfate Treatment of CB on PA Step Viral Clearance

This example demonstrated that the dextran sulfate treatment of CB can improve PA step viral clearance capability. Two model viruses, amphotropic murine leukemia viru (A-MuLV) and porcine parvovirus (PPV), were used for the study. Table 4 summarized the characteristics of these viruses.

TABLE 4
Summary of characteristics of viruses used in viral clearance study

| | | | | Approx. | |
|---|---|---|---|---|---|
| Virus | Virus Family | Envelope | Genome | Size (nm) | Shape |
| A-MuLV | Retroviridae | Yes | RNA | 80-1130 | Spherical |
| PPV | Parvoviridae | No | DNA | 18-26 | Icosahedral |

Fc-A cell culture CB was used in this study. Dextran sulfate final concentration of 0.1 g/g _{Protein} was used in the CB+DS run PA load material. CB without dextran sulfate was used as control PA load material. For each virus, the PA load material was spiked with 5% v/v of the appropriate stock virus solution. The PA run, sampling, and virus testing were conducted according to a protocol. The results of the virus clearance study were summarized in Table 5. The results show that dextran sulfate treated CB as PA load material achieved higher LRV than untreated CB.

TABLE 5
Viral Clearance Study Log Reduction Value (LRV)

| | CB as PA load material | | CB + DS as | |
|---|---|---|---|---|
| | (log10 adjusted | | PA load material | |
| | titer (PFU)) | | (log10 adjusted titer (PFU)) | |
| Process Steps | A-MuLV | PPV | A-MuLV | PPV |
| Load material | 7.37 | 10.11 | 8.37 | 10.03 |
| control | | | | |
| FT | 6.93 | 8.58 | 8.30 | 8.66 |
| Wash 1 | 5.62 | 6.92 | 6.34 | 6.50 |
| Wash 2 | 6.57 | 6.67 | <5.97 | 6.63 |
| Elution | 4.49 | 9.86 | <5.08 | 9.06 |
| LRV | 3.08 | 0.25 | >3.29 | 0.97 |

### Example 6

### Experimental Material and Method

CHO cells expressing either a monoclonal antibody or an Fc fusion protein were grown in a fed batch culture for 10-14 days. Cells were removed from cell culture harvest either by centrifugation or depth filtration. The clarified bulk (CB) was used for experiments. Experiments were conducted at room temperature unless otherwise noted.

Dextran sulfate (500 kDa, Product No. 31395) was purchased from Sigma (St. Louis, Mo.). 10 g/L dextran sulfate stock solution was prepared by dissolving into DI water. In each case, the stock solution was added to CB or protein A wash buffer to achieve the target dextran sulfate final concentration.

MabSelect™ Protein A resin was from GE Healthcare (Uppsala, Sweden). All chromatographic experiments were performed either on AKTA Explorer 100 or AKTA pilot chromatographic system from GE Healthcare (Uppsala, Sweden)

### EQUIVALENTS

Those skilled in the art will recognize or be able to ascertain using no more than routine experimentation, many equivalents of the specific embodiments of the invention described herein. Such equivalents are intended to be encompassed by the following claims.

### INCORPORATION BY REFERENCE

All patents, pending patent applications, and other publications cited herein are hereby incorporated by reference in their entireties.

1. A method of purifying a protein of interest from a mixture which comprises the protein of interest and one or more contaminants, comprising:
a) adding a dextran polymer to the mixture under conditions suitable for the dextran polymer to bind to one or more contaminants, thereby to form a second mixture;
b) subjecting the second mixture to an affinity chromatography;
c) contacting the affinity chromatography with a wash solution; and
b) recovering the protein of interest in an elution solution, thereby purifying the protein of interest.

2. A method of purifying a protein of interest from a mixture which comprises the protein of interest and one or more contaminants, comprising:
a) subjecting the mixture to an affinity chromatography;
b) contacting the affinity chromatography with a wash solution which comprises a dextran polymer, under conditions suitable for the dextran polymer to bind to one or more contaminants; and
c) recovering the protein of interest in an elution solution, thereby purifying the protein of interest.

3. The method of claim 1, wherein the contaminants are selected from host cell proteins, host cell metabolites, host cell constitutive proteins, nucleic acids, enzymes, endotoxins, viruses, product related contaminants, lipids, media additives and media derivatives, protein aggregates, chromatin, cell culture additives.

4. The method of claim 1, wherein said dextran polymer is selected from dextran, dextran sulfate, dextran sulfate sodium salt, DEAE-dextran hydrochloride.

5. The method of claim 4, wherein the molecular weight of dextran polymer ranges from 8 kDa to 500 kDa.

6. The method of claim 1, wherein the mixture is selected from a cell culture, a harvested cell culture fluid, a cell culture supernatant, a conditioned cell culture supernatant, a cell lysate, and a clarified bulk.

7. The method of claim 6, wherein the cell culture is a mammalian cell culture or a microbial cell culture.

8. The method of claim 6, wherein the cell culture is a Chinese Hamster Ovary (CHO) cell culture.

9. The method of claim 1, wherein the mixture comprises a feedstock.

10. The method of claim 6, wherein the mixture comprises cell culture media into which the protein of interest is secreted.

11. The method of claim 6, wherein the cell culture is in a bioreactor.

12. The method of claim 1, wherein the affinity chromatography is a Protein A chromatography.

13. The method of claim 1, further comprising subjecting the elution solution to a second chromatography.

14. The method of claim 13, wherein the second chromatography is selected from the group consisting of ion exchange, hydrophobic interaction, mimetic, and mixed mode.

15. The method of claim 1, wherein the protein of interest is an antibody or an Fc fusion protein.

16. The method of claim 15, wherein the antibody is a monoclonal antibody.

17. The method of claim 1, wherein the concentration of the dextran polymer is between about 0.01 and about 1 g/g protein in the mixture.

18. The method of claim 1, wherein the pH of the mixture is between about 6.5 and about 8.5.

19. The method of claim 2, wherein the concentration of the dextran polymer is between about 0.05 and about 2 g/L in the wash solution.

20. The method of claim 2, wherein the pH of the wash solution is between about 5.0 and about 10.0.
